(19) United States
(12) Patent Application Publication
(10) Pub. No.: US 2010/0331574 A1
(43) Pub. Date: Dec. 30, 2010

(54) HYDROXYAPATITE WITH SILVER SUPPORTED ON THE SURFACE THEREOF

(76) Inventors: Kiyotomi Kaneda, Osaka (JP); Noritsugu Yamasaki, Hyogo (JP)

Correspondence Address: BIRCH STEWART KOLASCH & BIRCH, PO BOX 747, FALLS CHURCH, VA, 22040-0747, US

(21) Appl. No.: 12/919,660

(22) PCT Filed: Mar. 3, 2009
(86) PCT No.: PCT/JP2009/000945

§ 371 Date: Aug. 26, 2010

(30) Foreign Application Priority Data
Mar. 6, 2008 (JP) 2008-055925
Jan. 8, 2009 (JP) 2009-002848

(51) Int. Cl. C07C 231/06; B01J 27/18

(52) U.S. Cl. 564/126; 502/208; 977/775

(57) ABSTRACT

An object of the present invention is to provide a hydroxyapatite with silver supported on the surface thereof, a new compound useful as a catalyst for the reaction of producing an amide compound by hydration of the corresponding nitrile compound.

The hydroxyapatite with silver supported on the surface thereof according to the present invention is obtained by supporting zero-valent Ag on the surface of a hydroxyapatite. Also provided are a hydroxyapatite with silver supported on the surface thereof used as a catalyst, and a method for producing an amide compound, comprising producing the amide compound by hydration of the corresponding nitrile compound in the presence of a hydroxyapatite with silver supported on the surface thereof having zero-valent Ag supported on the surface of the hydroxyapatite.

### TECHNICAL FIELD

The present invention relates to a new compound, a hydroxyapatite with silver supported on the surface thereof, and a method of producing amide compounds by using the hydroxyapatite with silver supported on the surface thereof.

### BACKGROUND ART

A nitrile gives a carboxylic acid and an amine when subjected to complete hydrolysis. If the reaction condition is selected properly, the intermediate amide compound is produced. The amide compounds thus obtained are useful, for example, as raw materials and intermediates for engineering plastics, synthetic detergents, lubricating oils and others.

Examples of known methods for producing amide compounds, which are useful as described above, include neutral hydrolysis, acidic hydrolysis, alkali hydrolysis, use of a biological catalyst and the like. The neutral hydrolysis method is a method of obtaining an amide compound by agitating a solution of a nitrile in dichloromethane with active manganese dioxide at room temperature (see, for example, Patent Document 1). However, the yield was still not sufficiently satisfactory.

The acidic hydrolysis method is a method of obtaining an amide compound by heating a nitrile with an acid such as hydrochloric acid, sulfuric acid or polyphosphoric acid. However, it was generally disadvantageous that the hydrolytic reaction of aromatic nitriles are slower. The alkali hydrolysis method was also disadvantageous in that the reaction easily proceeded to yield a carboxylic acid, making it difficult to obtain the intermediate amide compound.

The methods of using a biological catalyst include, for example, a method of producing amide compounds by using a microbe having enzyme activity. The method is advantageous for example in that the reaction condition is milder, enabling simplification of the reaction process, or the purity of the reaction product is higher because the by-products are formed in smaller amounts and thus, it has been used recently in production of many compounds (see, for example, Patent Document 2). Although the aqueous solution of an amide compound prepared by using microbe is a high-purity reaction solution, as the amide compound is contained at higher concentration in the reaction solution, the solution resulted in foaming easilier, possibly causing troubles in the following steps of: concentration, distillation, crystallization and polymerization and the like. In addition, the reaction condition suitable for microbial reactions is restricted and thus, the production of an amide compound by microbe was not sufficiently satisfactory from the point of its yield. Furthermore, the microbial production was also disadvantageous in that the microbe could not be used for the reactions many times repeatedly.

Thus, there existed a need for a catalyst allowing easy and efficient production of an amide compound by hydration of the corresponding nitrile compound.

Meanwhile, metal nanoparticles (NPs),which reside in the size range between bulk and monomeric metal species, are applied in a wide range of technologies, from electronic, optic and magnetic devices, to advanced catalytic materials. Currently, metal NP catalysts are receiving much attention for use in organic syntheses under liquid-phase conditions. For example, gold NPs have been shown to facilitate catalysis in many organic reactions. On the other hand, there have been few studies on the prominent catalytic activity of Ag NPs for other organic reactions, except for the gas-phase epoxidation of ethylene.

### Patent Document 1: Japanese Unexamined Patent Application No. 9-104665

### Patent Document 2: Japanese Unexamined Patent Application No. 11-123098

### DISCLOSURE OF THE INVENTION

### Problems to be Solved by the Invention

An object of the present invention is to provide a new compound, a hydroxyapatite with silver supported on the surface thereof, useful as a catalyst.

Another object of the present invention is to provide a method of producing amide compounds easily and efficiently by using the hydroxyapatite with silver supported on the surface thereof.

Yet another object of the present invention is to provide a hydroxyapatite carrying metal nanoparticle silver supported thereon.

Still another object of the present invention is to provide a method for producing amide compounds easily and efficiently by using the hydroxyapatite with metal nanoparticle silver supported on the surface thereof.

### Means to Solve the Problems

After intensive studies to solve the problems above, the inventors have found that a hydroxyapatite with silver supported on the surface thereof showed high catalytic activity and completed the present invention.

Moreover, the inventors have focused on the catalytic potential of Ag NPs, and found that supported Ag NPs show high catalytic activity for the dehydrogenation of alcohols and the selective oxidation of silanes to silanols using water under liquid-phase conditions.

Thus, the present invention provides a hydroxyapatite with silver supported on the surface thereof having zero-valent Ag supported on the surface of a hydroxyapatite.

The hydroxyapatite with silver supported on the surface thereof is preferably used as a catalyst.

The present invention also provides a method for producing amide compounds, comprising producing the amide compound by hydration of the corresponding nitrile compound in the presence of a hydroxyapatite with silver supported on the surface thereof having zero-valent Ag supported on the surface of a hydroxyapatite.

The present invention further provides a hydroxyapatite with silver supported on the surface thereof having nanoparticle metal zero-valent Ag . supported on the surface of a hydroxyapatite.

The present invention still further provides a method for producing amide compounds comprising producing amide compounds by hydration of the corresponding nitrile compound in the presence of a hydroxyapatite with silver supported on the surface thereof having nanoparticle metal zero-valent Ag supported on the surface of a hydroxyapatite.

### Advantageous Effect of the Invention

The hydroxyapatite with silver supported on the surface thereof according to the present invention can be prepared easily and shows high activity in the reaction of producing an amide compound by hydration of the corresponding nitrile compound. In addition, the hydroxyapatite with silver supported on the surface thereof according to the present invention, which is solid, can be reused easily and in particular, can be reused repeatedly while keeping high activity without particular need for additional regeneration treatment.

It is possible through the method of the present invention to obtain an amide compound by hydration of the corresponding nitrile compound at high yield.

The present invention demonstrates that hydroxyapatite (HAP)-supported Ag NPs (AgHAP) can catalyze hydration of nitriles to amide in water with high efficiency. Hydration of nitriles into the corresponding amides is of great importance in organic syntheses, because amides are versatile synthetic intermediates used in the production of pharmacological products, polymers, detergents, lubricants and drug stabilizers. However, traditional catalyst systems have required organic solvents in the presence of homogeneous strong acid and base catalysts, which causes overhydrolysis of amides into undesirable carboxylic acids, and the formation of a large amount of salts after neutralization of the catalysts. Therefore, much effort has been expended on the development of effective metal catalysis for the hydration of nitriles. This hydration method, using a reusable Ag catalyst under neutral conditions with water as the solvent, can make a significant contribution to establish a more environmentally-benign and industrially-acceptable process.

### BEST MODE FOR CARRYING OUT THE INVENTION

[Hydroxyapatite with Silver Supported on the Surface Thereof]

The hydroxyapatite with silver supported on the surface thereof according to the present invention has zero-valent Ag supported on the surface of a hydroxyapatite.

The hydroxyapatite is, for example, a compound represented by the following Formula (1):

Ca_{10-Z}(HPO₄)_{Z}(PO₄)_{6-Z}(OH)_{2-Z}.nH₂O (1)

wherein, Z is a number satisfying 0≦Z≦1, and n is a number of 0 to 2.5.

The hydroxyapatite can be prepared, for example, by a wet production method. The wet production method is specifically a method of precipitating a hydroxyapatite in a buffer solution by adding a calcium solution and a phosphate solution at a molar concentration ratio of 10:6 into a buffer solution having a pH kept at a particular value of 7.4 or more dropwise over an extended period and collecting the precipitated hydroxyapatite.

An example of the hydroxyapatite favorably used in the present invention is “Tricalcium phosphate (trade name)”, manufactured by Wake Pure Chemical Industries, Ltd.

The method of supporting zero-valent Ag on the hydroxyapatite surface is, for example, a method of making a silver compound adsorbed on the surface of a hydroxyapatite by mixing a silver compound solution with the hydroxyapatite, agitating the mixture and reducing the silver compound carrying hydroxyapatite. Examples of the silver compounds for use include silver salts such as chloride, bromide, iodide, carbonate, nitrate, sulfate and phosphate; silver complexes and the like.

The solvent is not particularly limited, if it can dissolve the silver compound, and examples thereof include water, acetone, alcohols and the like. The concentration of the silver compound in the solution during supporting Ag is not particularly limited, and can be selected, for example, in the range of 0.1 to 1000 mM. The temperature during agitating may be selected, for example, in the range of 20 to 150° C., but agitation may be performed normally at room temperature. The Ag content of hydroxyapatite with silver supported on the surface thereof is not particularly limited, but may be selected, for example, in the range of 0.01 to 10 mmol, preferably 0.05 to 0.5 mmol, with respect to 1 g of the hydroxyapatite. The agitating time may vary according to the temperature during agitating, but may be selected, for example, in the range of 1 to 360 minutes, preferably 5 to 90 minutes. After agitation, the resulting hydroxyapatite may be washed, as needed, with water, an organic solvent or the like, dried and subjected to reduction treatment, to give a hydroxyapatite with silver supported on the surface thereof according to the present invention.

Examples of the reducing agents used in the reduction treatment include borohydride complex compounds such as sodium borohydride (NaBH₄), lithium borohydride (LiBH₄) and potassium borohydride (KBH₄), hydrazine, hydrogen (HA silane compounds such as trimethylsilane, hydroxy compounds and the like. The hydroxy compounds include alcoholic compounds such as primary and secondary alcohols. Alternatively, the hydroxy compound may have multiple hydroxyl groups and thus, may be a monohydric alcohol, a dihydric alcohol, a polyhydric alcohol or the like.

Borohydride complex compounds are preferable, and potassium borohydride (KBH₄) is particularly preferable, among the reducing agents of the present invention. The hydroxyapatite with silver supported on the surface thereof obtained by reduction with potassium borohydride (KBH₄) often has smaller average diameter of the supported Ag particles and thus has increased specific surface area, showing drastically improved catalytic activity.

The hydroxyapatite with silver supported on the surface thereof according to the present invention can be used as a catalyst. Examples of the reactions catalyzed thereby include amide compound forming reactions by hydration of the respective corresponding nitrile compounds, silanol compound-forming reactions by oxidation of a silane compound, and the like.

### [Production of Amide Compounds]

The method of producing amide compounds according to the present invention is characterized by producing an amide compound by hydration of the corresponding nitrile compound in the presence of the hydroxyapatite with silver supported on the surface thereof according to the present invention carrying Ag supported thereon described above. It is possible by the method of the present invention to produce an amide compound by hydration of the corresponding nitrile compound at high yield.

The nitrile compound according to the present invention is represented by the General Formula (2):

R—C≡N (2)

wherein, R represents an organic group.

The organic group R is not particularly limited, if it is not a group inhibiting the reaction (for example, if it is a group non-reactive under the reaction condition of the present method), and examples thereof include hydrocarbon groups, heterocyclic groups and the like. The hydrocarbon and heterocyclic groups also include substituted hydrocarbon and heterocyclic groups.

The hydrocarbon groups R include aliphatic hydrocarbon groups, alicyclic hydrocarbon groups, aromatic hydrocarbon groups and groups in combination of these groups. Examples of the aliphatic hydrocarbon groups include alkyl groups having approximately 1 to 20 carbon atoms (preferably 1 to 10, more preferably 1 to 3) such as methyl, ethyl, propyl, isopropyl, butyl, isobutyl, s-butyl, t-butyl, pentyl, hexyl, decyl and dodecyl groups; alkenyl groups having approximately 2 to 20 carbon atoms (preferably 2 to 10, more preferably 2 to 3) such as vinyl, allyl and 1-butenyl groups; alkynyl group having approximately 2 to 20 carbon atoms (preferably 2 to 10, more preferably 2 to 3) such as ethynyl and propynyl groups; and the like.

Examples of the alicyclic hydrocarbon groups include approximately 3- to 20-membered (preferably 3- to 15-membered, more preferably 5- to 8-membered) cycloalkyl groups such as cyclopropyl, cyclobutyl, cyclopentyl, cyclohexyl and cycloocty groups 1; approximately 3- to 20-membered (preferably 3 to 15-membered, more preferably 5- to 8-membered) cycloalkenyl groups such as cyclopentenyl and cyclohexenyl groups; bridged cyclic hydrocarbon groups such as perhydronaphthalen-1-yl, norbornyl, adamantyl and tetracyclo[4.4.0.1^{2,5}.1^{7,10}]-dodecan-3-yl groups; and the like. Examples of the aromatic hydrocarbon groups include aromatic hydrocarbon groups having approximately 6 to 14 carbon atoms (preferably 6 to 10) such as phenyl and naphthyl groups.

Examples of the hydrocarbon group in combination of the aliphatic and alicyclic hydrocarbon groups include cycloalkyl-alkyl groups (for example, C₃₋₂₀ cycloalkyl-C₁₋₄ alkyl group and others) such as cyclopentylmethyl, cyclohexylmethyl and 2-cyclohexylethyl groups, and the like. Examples of the hydrocarbon groups in combination of the aliphatic and aromatic hydrocarbon groups include aralkyl groups (for example, C₇₋₁₈ aralkyl groups and others), alkyl-substituted aryl groups (for example, phenyl or naphthyl groups substituted with approximately 1 to 4 C₁₋₄ alkyl groups), aryl-substituted C₂₋₁₀ alkenyl groups (for example, 2-phenylvinyl group) and the like.

The hydrocarbon group R is preferably a C₁₋₁₀ alkyl group, a C₂₋₁₀ alkenyl group, an aryl-substituted C₂₋₁₀ alkenyl group, a C₂₋₁₀ alkynyl group, a C₃₋₁₅ cycloalkyl group, a C₆₋₁₄ aromatic hydrocarbon group, a C₃₋₁₅ cycloalkyl-C₁₋₄ alkyl group, a C₇₋₁₄ aralkyl group, a phenyl or naphthyl group substituted with approximately 1 to C₁₋₄ alkyl groups, or the like.

The hydrocarbon group may have various substituent groups, such as halogen atoms, oxo group, hydroxyl group, substituted oxy groups (such as alkoxy, aryloxy, aralkyloxy and acyloxy), carboxyl group, substituted oxycarbonyl groups (such as alkoxycarbonyl, aryloxycarbonyl and aralkyloxycarbonyl), substituted or unsubstituted carbamoyl group, cyano group, nitro group, acyl group, substituted or unsubstituted amino groups, sulfa group and heterocyclic groups. The hydroxyl and carboxyl groups may be protected with a protecting group conventionally used in the field of organic synthesis. In addition, the ring in the alicyclic or aromatic hydrocarbon group may be fused with an aromatic or non-aromatic heterocyclic ring.

The heterocyclic rings constituting the heterocyclic group R described above include aromatic and non-aromatic heterocyclic rings. Examples of the heterocyclic rings include heterocyclic rings containing one or more oxygen atoms as heteroatoms (including five-membered rings such as furan, tetrahydrofuran, oxazole, isoxazole and γ-butylolactone; six-membered rings such as 4-oxo-4H-pyran, tetrahydropyran and morpholine; fused rings such as benzofuran, isobenzofuran, 4-oxo-4H-chromene, chromane and isochromane; and bridged rings such as 3-oxatricyclo[4.3.1.1^{4,8}]-undecan-2-one and 3-oxatricyclo[4.2.1.0^{4,8}]-nonan-2-one), heterocyclic rings containing one or more sulfur atoms as heteroatom (including five-membered rings such as thiophene, thiazole, isothiazole and thiadiazole; six-membered rings such as 4-oxo-4H-thiopyran; and fuse rings such as benzothiophene), heterocyclic rings containing one or more nitrogen atoms as heteroatoms (including five-membered rings such as pyrrole, pyrrolidine, pyrazole, imidazole and triazole; six-membered rings such as pyridine, pyridazine, pyrimidine, pyrazine, piperidine and piperazine; fused rings such as indole, indoline, quinoline, acridine, naphthyridine, quinazoline and purine) and the like. The heterocyclic group may have, in addition to one or more of the substituent groups to the hydrocarbon group, one or more additional substituents such as alkyl groups (including C₁₋₄ alkyl groups such as methyl and ethyl), cycloalkyl groups, aryl groups (such as phenyl and naphthyl) and the like,

Favorable groups R include, hydrocarbon groups (C₆₋₁₄ aromatic hydrocarbon groups, C₇₋₁₄ aralkyl groups, phenyl or naphthyl groups substituted with approximately 1 to 4 C₁₋₄ alkyl groups, aryl-substituted C₂₋₁₀ alkenyl groups, C₂₋₁₀ alkenyl groups and the like); aromatic heterocyclic rings having one or more oxygen, sulfur and nitrogen atoms as heteroatoms; and the like.

The nitrile compounds according to the present inventions may include, for example, benzonitrile, p-cyanotoluene, m-cyanotoluene, o-cyanotoluene, p-chlorobenzonitrile, m-chlorobenzonitrile, o-chlorobenzonitrile, 3-phenylacrylonitrile, 3-cyanopyridine, 2-cyanothiophene, 2-chloro-3-cyanopyridine, 2-cyanopyrazine, 2-cyanofuran, 2-cyano-5-methylfuran, 3-cyanoquinoline, acrylonitrile, methacrylonitrile, acetonitrile, propionitrile, butanenitrile, hexanenitrile, 2-naphthonitrile, p-nitrobenzonitrile, p-acetylbenzonitrile, p-fluorobenzonitrile and the like.

The nitrile compound may be converted to the corresponding amide compound by hydration thereof in the presence of the hydroxyapatite with silver supported on the surface thereof. The amount of water used in the hydration reaction is, for example, approximately 1 to 10 moles, with respect to 1 mole of the nitrile compound. Water may be used in large excess.

The reaction can be carried out, for example, by mixing the nitrile compound with a hydroxyapatite with silver supported on the surface thereof and agitating the mixture. The amount of the hydroxyapatite with silver supported on the surface thereof used is not particularly limited, but selected, for example, in the range, as silver, of 0.001 to 1 mol, preferably 0.001 to 0.1 mol, particularly preferably 0.01 to 0.1 mol, with respect to 1 mole of the nitrile compound. The reaction may be carried out in liquid or gas phase. When processability and others are taken into consideration, the reaction is preferably carried out in liquid phase in the present invention.

The reaction may be carried out in the presence or absence of solvent. The solvent is not particularly limited, if it does not inhibit the reaction, and one of conventional commonly used solvents may be used as properly selected. Examples thereof include water; fluorochemical solvents such as trifluorotoluene, fluorobenzene and fluorohexane; aromatic hydrocarbons such as benzene, toluene, xylene, chlorobenzene and nitrobenzene; aliphatic hydrocarbons such as pentane, hexane, heptane, octane, cyclohexane and methylcyclohexane; ethers such as 1,2-dioxane, 1,3-dioxane, 1,4-dioxane, tetrahydrofuran, tetrahydropyran, diethylether and dimethylether; amides such as acetamide, dimethylacetamide, dimethylformamide, diethylformamide and N-methylpyrrolidone; esters such as ethyl acetate, propyl acetate and butyl acetate; the mixtures thereof and the like. In particular, polar solvents are preferable, and in particular, water, which is highly polar, can be used favorably in the present invention.

The reaction may be carried out under normal pressure or under pressure. The reaction temperature is not particularly limited and may be selected according to the kinds of the nitrile compound as the raw materials and the solvent used, but may be selected, for example, in the range of 0 to 250° C., more preferably 60 to 200° C., particularly preferably 100 to 200° C.

The reaction time is not particularly limited and may be selected according to the kinds of the nitrile compound as the raw materials and the solvent used, but may be preferably selected, for example, in the range of 0.1 to 200 hours, more preferably 0.1 to 50 hours. The reaction may be carried out by a conventional method, for example, batchwise, semi-batchwise or continuously. After reaction, the reaction product can be separated and purified, for example, by a separation means such as a filtration, concentration, distillation, extraction, crystallization, recrystallization, adsorption or column chromatography or in combination of these separation means.

The hydroxyapatite with silver supported on the surface thereof according to the present invention, which carries silver tightly supported on the hydroxyapatite surface, does not release silver into the reaction solution. Thus, after reaction and recovery by operation such as filtration or centrifugation, the hydroxyapatite with silver supported on the surface thereof can be used as a catalyst for hydration of nitrile compounds, as it is or after additional washing process as needed with water, organic solvent or the like. Even when the hydroxyapatite with silver supported on the surface thereof is used in reaction repeatedly, it retains its favorable catalytic activity consistently and allows production of the corresponding amide compound at high yield.

### EXAMPLES

Hereinafter, the present invention will be described more in detail with reference to Examples, but it should be understood that the present invention is not restricted by these Examples.

### Preparative Example 1

A 200-mL round-bottomed flask was charged with AgNO₃ (1.0 mmol) and water (150 mL) to give an aqueous silver solution. Then 2.0 g of hydroxyapatite (Tricalcium phosphate, manufactured by Wako Pure Chemical Industries, Ltd) was added thereto; and the mixture was agitated under air atmosphere at room temperature (25° C.) for 6 hours. The mixture was filtered under reduced pressure after agitation, washed with deionized water (1 L) and dried under vacuum for 24 hours, to give an Ag(I)/hydroxyapatite catalyst (Ag content: 0.3 mmol/g).

To a 200-mL round-bottomed flask was added Water (150 mL) and KBH₄ (9 mmol) to give a homogeneous solution; the Ag(I)/hydroxyapatite catalyst obtained (1.8 g) was added thereto; and the mixture was agitated under argon atmosphere at room temperature (25° C.) for 1 hour. The mixture was filtered under reduced pressure after agitation, washed with deionized water (1 L) and dried under vacuum for 24 hours, to give an Ag(0)/hydroxyapatite catalyst (Ag content: 0.3 mmol/g).

### Preparative Example 2

An Ag(I)/hydroxyapatite catalyst was prepared in a manner similar to Preparative Example 1.

To a 200-mL round-bottomed flask was added Water (150 mL) and hydrazine (9 mmol) to give a homogeneous solution; the Ag(I)/hydroxyapatite catalyst obtained (1.8 g) was added thereto; and the mixture was agitated under argon atmosphere 60° C. for 1 hour, The mixture was filtered under reduced pressure after agitation, washed with deionized water (1 L) and dried under vacuum for 24 hours, to give and Ag(0)/hydroxyapatite catalyst (Ag content: 0.3 mmol/g).

### Preparative Example 3

A 200-mL round-bottomed flask was charged with AgNO₃ (1.0 mmol) and water (150 mL) to give an aqueous silver solution; 2.0 g of fluoroapatite (trade name “Apatite FAP, hexaclinic”, manufactured by Wako Pure Chemical Industries, Ltd) was added thereto; and the mixture was agitated at room temperature (25° C.) for 6 hours. The mixture was then washed with deionized water and dried under vacuum at room temperature (25° C.) for 24 hours, to give an Ag(I)/fluoroapatite catalyst.

To a 200-mL round-bottomed flask was added Water (150 mL) and KBH₄ (9 mmol) to give a homogeneous solution; the Ag(I)/fluoroapatite catalyst obtained (1.8 g) was added thereto; and the mixture was agitated under argon atmosphere at room temperature (25° C.) for 1 hour. The mixture was filtered under reduced pressure after agitation, washed with deionized water (1 L) and dried under vacuum for 24 hours, to give an Ag(0)/fluoroapatite catalyst (Ag content: 0.1 mmol/g).

### Preparative Example 4

A 200-mL round-bottomed flask was charged with AgNO₃ (1.0 mmol) and water (150 mL) to give an aqueous silver solution; 1.5 g of γ-ZrP (trade name “CZP-200”, manufactured by Daiichi Kigenso Kagaku Kogyo Co., Ltd.) was added thereto; and the mixture was agitated at room temperature (25° C.) for 6 hours. The mixture was then washed with deionized water and dried under vacuum at room temperature (25° C.) for 24 hours, to give an Ag(I)/γ-ZrP catalyst.

To a 200-mL round⁻bottomed flask was added KBH₄ (9 mmol) and water (150 mL) to give a homogeneous solution; and the Ag(I)/γ-ZrP catalyst obtained (1.8 g) was added thereto; and the mixture was agitated under argon atmosphere at room temperature (25° C.) for 1 hour. The mixture was filtered under reduced pressure after agitation, washed with deionized water (1 L), and dried under vacuum for 24 hours, to give an Ag(0)/γ-ZrP catalyst (Ag content: 0.5 mmol/g).

### Preparative Example 5

A 200 mL round-bottomed flask was charged with AgNO₃ (1.0 mmol) and water (150 mL) to give an aqueous silver solution; 2.0 g of HT (trade name “Tomita AD500NS”, manufactured by Tomita Pharmaceutical Co., Ltd.) was added thereto; and the mixture was agitated at room temperature (25° C.) for 6 hours. The mixture was then washed with deionized water and dried under vacuum at room temperature (25° C.) for 24 hours, to give an Ag(I)/HT catalyst.

To a 200-mL round-bottomed flask was added KBH₄ (9 mmol) and water (150 mL) to give a homogeneous solution; and the Ag(I)/HT catalyst (1.8 g) obtained was added thereto; and the mixture was agitated under argon atmosphere at room temperature (25° C.) for 1 hour. The mixture was filtered under reduced pressure after agitation, washed with deionized water (1 L), and dried under vacuum for 24 hours, to give an Ag(0)/HT catalyst (Ag content: 0.2 mmol/g).

### Example 1

0.1 g of the Ag(0)/hydroxyapatite catalyst (Ag: 0.03 mmol) obtained in Preparative Example 1, 3 mL of water and 0.1 g of benzonitrile (1.0 mmol) were placed in a glass pressure reaction tube, and the mixture was agitated under air atmosphere at 140° C. for 2 hours, to give benzamide at a conversion rate of 93% and a yield of 90%.

### Example 2

0.1 g of the Ag(0)/hydroxyapatite catalyst (Ag: 0.03 mmol) obtained in Preparative Example 2, 3 mL of water and 0.1 g of benzonitrile (1.0 mmol) were placed in a glass pressure reaction tube, and the mixture was agitated under air atmosphere at 140° C. for 2 hours, to give benzamide at a conversion rate of 59% and a yield of 60%.

Examples 3 to 19 were carried out in a manner similar to Example 1, except that the raw nitrile compound and the reaction temperature were altered. Results are summarized in the following Tables 1 and 2.

TABLE 1

| | | Reaction | | Conversion | |
|---|---|---|---|---|---|
| Example No. | Nitrile compound | temperature (° C.) | Reaction time (h) | rate (%) | Yield (%) |
| 3 | 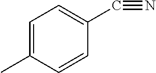 | 160 | 2 | 96 | 96 |
| 4 | 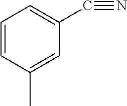 | 180 | 2 | 99 | 99 |
| 5 | 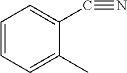 | 180 | 2 | 47 | 47 |
| 6 | 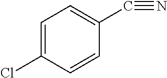 | 140 | 6 | 99 | 99 |
| 7 | 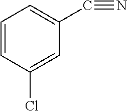 | 160 | 2 | 99 | 99 |
| 8 | 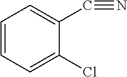 | 180 | 2 | 99 | 99 |
| 9 | 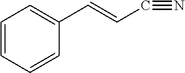 | 180 | 6 | 84 | 84 |
| 10 | 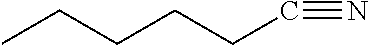 | 180 | 6 | 2 | 2 |

TABLE 2

| | | Reaction | | Conversion | |
|---|---|---|---|---|---|
| Example No. | Nitrite compound | temperature (° C.) | Reaction time (h) | rate (%) | Yield (%) |
| 11 12 | 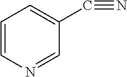 | 140 80 | 0.5 24 | 95 92 | 95 98 |
| 13 14 | 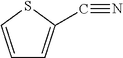 | 140 80 | 0.3 24 | 98 99 | 98 94 |
| 15 16 | 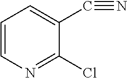 | 140 140 | 0.17 0.5 | 56 80 | 28 54 |
| 17 18 19 | 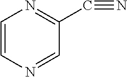 | 140 80 60 | 0.17 48 24 | 99 94 83 | 99 94 79 |

### Example 20

After the reaction in Example 1, the Ag(0)Ihydroxyapatite catalyst after use was recovered by filtration of the reaction solution, and the recovered Ag(0)/hydroxyapatite catalyst was washed with water, to give a regenerated Ag(0)/hydroxyapatite catalyst.

The reaction was repeated in a manner similar to Example 1, except that the regenerated Ag(0)/hydroxyapatite catalyst was used, to give benzamide at a yield of 88%.

### Example 21

After the reaction in Example 21, the regenerated Ag(0)/hydroxyapatite catalyst after use was recovered again by filtration of the reaction solution and the recovered Ag(0)/hydroxyapatite catalyst was washed with water, to give a twice-regenerated Ag(0)/hydroxyapatite catalyst.

The reaction was repeated in a manner similar to Example 1 except that the twice-regenerated Ag(0)/hydroxyapatite catalyst was used, to give benzamide at a yield of 87%.

### Comparative Example 1

0.1 g of hydroxyapatite (Tricalcium phosphate, manufactured by Wako Pure Chemical Industries, Ltd), 3 mL of water and 0.1 g of benzonitrile (1.0 mmol) were placed in a glass pressure reaction tube and the mixture was agitated under air atmosphere at 140° C. for 2 hours, only to give no benzamide.

### Comparative Example 2

0.1 g of the Ag(0)/fluoroapatite catalyst obtained in Preparative Example 3 (Ag: 0.01 mmol), 3 mL of water and 0.1 g of benzonitrile (1.0 mmol) were placed in a glass pressure reaction tube and the mixture was agitated under air atmosphere at 140° C. for 2 hours, to give benzamide at a conversion rate of 39% and a yield of 32%.

### Comparative Example 3

0.1 g of the Ag(0)/γ-ZrP catalyst obtained in Preparative Example 4 (Ag: 0.05 mmol), 3 mL of water and 0.1 g of benzonitrile (1.0 mmol) were placed in a glass pressure reaction tube and the mixture was agitated under air atmosphere at 140° C. for 2 hours, to give benzamide at a conversion rate of 18% and a yield of 11%.

### Comparative Example 4

0.1 g of the Ag(0)/HT catalyst obtained in Preparative Example 5 (Ag: 0.02 mmol), 3 mL of water and 0.1 g of benzonitrile (1.0 mmol) were placed in a glass pressure reaction tube and the mixture was agitated under air atmosphere at 140° C. for 2 hours, to give benzamide at a conversion rate of 46% and a yield of 40%.

Example 22. Nanoparticle silver-supported catalyst for production of an amide by selective hydration of the corresponding nitrile in water

AgHAP was synthesized as follows: 2.0 g of Ca₅(PO₄)₃(OH)(HAP) was soaked in a 150 mL aqueous solution of AgNO₃ (6.7×10⁻³ M) and agitated at room temperature for 6 h. The obtained slurry was filtered, washed and dried at room temperature under vacuum. Reduction with an aqueous solution of HBH₄ yielded HAP-supported AgHAP (Ag 3.3 wt %). The X-ray diffraction (XRD) peak positions of AgHAP were similar to those of the parent HAP, and transmission electron microscopy (TEM) showed that Ag NPs with a mean diameter of 7.6 nm and a narrow size distribution (standard deviation of 1.8 nm) were formed on the surface on the HAP substrate.

The catalytic activity of Ag⁰ NPs formed on different supports was tested for the hydration of benzonitrile (1) under aqueous conditions without organic solvents. AgHAP was an effective catalyst, affording benzamide as a sole product in a 99% yield (Table 3, entry 1). The use of Ag/TiO₂ in place of AgHAP showed a relatively high conversion of 1; however, benzoic acid was formed as a side product via over-hydrolysis of benzamide. Ag/MgO, Ag/SiO₂ and Ag/C were significantly less active. The hydration reaction did not proceed using HAP and Ag⁺HAP without a reduction treatment. After filtration of the reaction mixture containing AgHAP at a 40% conversion of 1, further agitating of the filtrate at 140° C. for 3 hours did not yield any addition products, and no Ag species was detected in the filtrate by inductively coupled plasma spectroscopy (ICP) analysis. These results show that the combination of Ag⁰ NPs with HAP is essential for efficient hydration, and the hydration proceeds at the Ag NPs on the surface of HAP. The scope of nitrile reactants for AgHAP-catalyzed hydration was surveyed. As exemplified in Table 3, AgHAP was efficient for the hydration of nitriles, except for alkyl nitriles (entries 14 and 15). Various benzonitrile derivatives were hydrated in high yields with over 99% selectivity for the corresponding amides (entries 1-12). The steric effect of ortho-substituted nitriles on the reaction rates was observed (entries 2 and 9). The hydration of cinnamonitrile proceeded to afford cinnamamide with an intact C═C double bond (entry 13). The hydration of various heteroaromatic nitriles was next carried out using the AgHAP catalyst, as summarized in Table 4. Remarkably, many of the heteroaromatic nitriles containing nitrogen, oxygen and sulfur atoms were effectively converted into the corresponding amides within only 1 hour, and no accompanying carboxylic acids were detected. For example, hydration of 2-cyanopyridine, 2-furancarbonitrile and 2-thiophenecarbonitrile afforded the corresponding amides in quantitative yields (entries 1, 5 and 7). Even a very water insoluble nitrile, such as 3-quinolinecarbonitrile, was also hydrated to 3-quinolinecarboxamide in a 95% yield (entry 4). It is notable that pyrazinecarbonitrile (2) was hydrated within only 10 min, and the corresponding pyrazinecarboxamide, which is used as a medicine for tuberculosis, was obtained in a 99% yield (entry 8), moreover, 2 was converted quantitatively even at 40° C. (entry 9). The AgHAP catalyst system was also applicable for scaled-up conditions; 2 (100 mmol; 10.5 g) was successfully converted to the amide (97% isolated yield; 12.0 g) and the turnover number (TOP) reached over 10000 (entry 10). To the best of our knowledge, such specifically enhanced reactivity of heteroaromatic nitriles compared with other nitriles has not been reported.

Furthermore, AgHAP was easily separated by centrifugation after hydration of 2, and could be reused four times for the hydration of 2 without loss of catalytic activity and selectivity (entries 11-14). Interactions between the AgHAP surface and nitriles were examined using Fourier transform infrared (FTIR) spectroscopy. 1,2-cyanopyridine and hexanenitrile were treated with AgHAP, respectively, and each absorption band assigned to a C≡N stretching vibration of the adsorbed nitriles was shifted to higher frequencies with respect to their liquid forms, which indicates side-on coordination of the nitrile groups on Ag NPs. Furthermore, the nitriles adsorbed onto AgHAP were also exposed to water vapor at 298 K. Time-resolved IR spectra showed that the nitrile band of 3 gradually decreased in intensity with the increase of a new band indicating CO═O stretching vibration. The production of amide was also confirmed by mass spectrometry, while the intensity of the nitrile IR band of 1 slightly decreased and that of 4 was hardly changed. The order of reactivity of the adsorbed nitriles with water vapor is 3>1>4, which is consistent with the results of catalytic hydration of the nitriles using AgHAP, as shown in Tables 3 and 4.

Without wishing to be bound by any particular theory, a possible mechanism involving the coordination of water and an aromatic nitrile on the AgHAP surface is proposed. Aromatic nitriles are strongly activated on Ag NPs of AgHAP through the dual activation of cyano and aromatic groups. Subsequently a nucleophilic OH from H₂O, which is generated on the Ag surface, easily attacks the proximal nitrile carbon atom to form the corresponding amide through an iminol transition state.

In conclusion, novel catalytic properties of Ag NPs for selective hydration of nitriles into amides were discovered. HAP-supported Ag NPs act as a highly active and reusable solid catalyst for the hydration of aromatic nitriles in water.

TABLE 3
Scope of nitriles for in AgHAP-catalyzed hydration^{a}

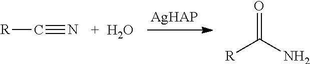

| | | | t | Yield | | | | t | Yield |
|---|---|---|---|---|---|---|---|---|---|
| Number | Reactant | | Time | (%)^{b} | Number | Reactive group | | (hour) | (%)^{b} |
| 1 | 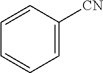 | | 3 | 99 (94) | 8^{d} | 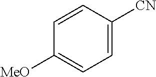 | | 2 | 97 (92) |
| 2^{c} | | o | 6 | 99 (94) | 9^{c} | | o | 6 | 47 |
| 3^{d} | 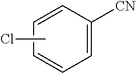 | m | 2 | 99 (94) | 10^{c} | 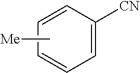 | m | 2 | 99 (96) |
| 4 | | p | 2 | 99 (96) | 11^{d} | | p | 2 | 96 (92) |
| 5 | 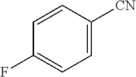 | | 2 | 99 (95) | 12 | 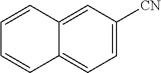 | | 6 | 98 (93) |
| 6 | 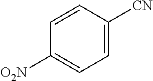 | | 1 | 99 (95) | 13^{c} | 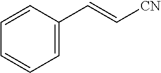 | | 6 | 84 (79) |
| 7 | 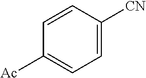 | | 1 | 99 (97) | 14^{c} | 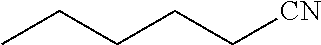 | | 6 | 2 |
| | | | | | 15^{c} | 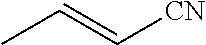 | | 6 | 2 |

^{a}Reaction conditions: reactant (1 mmol), AgHAP (0.1 g, Ag; 0.03 mmol), water (3 mL), 140° C.
^{b}The values in parenthesis are isolated yields
^{c}At 180° C.
^{d}At 160° C.

TABLE 4
Hydration of various heteroaromatic nitriles using AgHAP^{a}

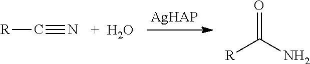

| | | t | Yield | | | t | Yield |
|---|---|---|---|---|---|---|---|
| Number | Reactant | (min) | (%)^{b} | Number | Reactive group | (min) | (%)^{b} |
| 1 | 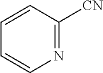 | 15 | 99 (94) | 6 | 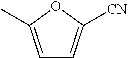 | 30 | 99 (95) |
| 2 | 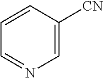 | 30 | 95 (93) | 7 | 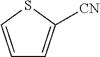 | 20 | 98 (96) |
| 3 | 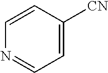 | 20 | 98 (94) | 8 | | 10 | 99 (96) |
| 4 | 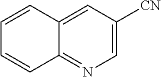 | 60 | 95 (91) | 9^{c} | | 2880 | 94 |
| 5 | 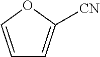 | 10 | 99 (95) | 10^{d} | | 2880 | 99 (97) |
| | | | | 11^{e} | 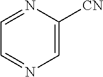 | 10 | 99 |
| | | | | 12^{f} | | 10 | 99 |
| | | | | 13^{g} | | 10 | 99 |
| | | | | 14^{h} | | 10 | 99 |

^{a}Reaction condition: reactant (1 mmol), AgHAP (0.1 g, Ag; 0.03 mmol), H₂O (3 mL), 140° C.
^{b}The values in parenthesis are isolated yields.
^{c}Reactant (0.5 mmol), 40° C.
^{d}Reactant (100 mmol), AgHAP (0.03 g, Ag; 0.009 mmol), H₂O (35 mL).
^{e}1st reuse,
^{f}2nd reuse,
^{g}3rd reuse,
^{h} 4th reuse.

What is claimed is:

1. A hydroxyapatite with silver supported on the surface thereof, comprising zero-valent Ag supported on the surface of the hydroxyapatite.

2. The hydroxyapatite with silver supported on the surface thereof according to claim 1 for use as a catalyst.

3. A method for producing an amide compound, comprising producing the amide compound by hydration of the corresponding nitrile compound in the presence of a hydroxyapatite with silver supported on the surface thereof having zero-valent Ag supported on the surface of the hydroxyapatite.

4. The hydroxyapatite with silver supported on the surface thereof according to claim 1 or 2, wherein the Ag is metal nanoparticles.

5. The method for producing an amide compound according to claim 3, wherein the Ag is metal nanoparticles.
